(19) United States
(12) Patent Application Publication
(10) Pub. No.: US 2023/0054405 A1
(43) Pub. Date: Feb. 23, 2023

(54) RHINITIS THERAPY APPARATUS AND METHOD FOR OPERATING THEREOF

(71) Applicant: Node Co., Ltd., Wongu-si (KR)

(72) Inventors: Jihoon PARK, Yongin-si (KR); Nara LEE, Wonju-si (KR)

(73) Assignee: Node Co., Ltd., Wonju-si (KR)

(21) Appl. No.: 17/532,341

(22) Filed: Nov. 22, 2021

(30) Foreign Application Priority Data
Aug. 18, 2021 (KR) 10-2021-0108863

(51) Int. Cl. A61N 2/02; A61N 2/00; G16H 40/67

(52) U.S. Cl. CPC A61N 2/02; A61N 2/004; G16H 40/67

(57) ABSTRACT

The present disclosure provides a rhinitis therapy apparatus, which includes a body configured to be able to cover a nose region of a user, and a magnetic field generator provided on the body and irradiating a magnetic field toward the nose region, in which the magnetic field generator includes a first coil guide, a second coil guide disposed to face the first coil guide, and a magnetic core disposed between the first coil guide and the second coil guide and including a coil wound thereon, and a method for operating the same.

### CROSS-REFERENCE TO RELATED APPLICATIONS

This application claims priority under 35 U.S.C §119 to Korean Patent Application No. 10-2021-0108863 filed in the Korean Intellectual Property Office on Aug. 18, 2021, the entire contents of which are hereby incorporated by reference.

### TECHNICAL FIELD

The present disclosure relates to a rhinitis therapy apparatus, and specifically, to a rhinitis therapy apparatus including a magnetic field generator including a structure capable of effectively irradiating a magnetic field to a nose region of a user, and a method for operating the same.

Specifically, the present disclosure relates to a rhinitis therapy apparatus including a body configured to be able to cover a nose region of a user, and a magnetic field generator provided on the body and irradiating a magnetic field toward the nose region, in which the magnetic field generator includes a first coil guide, a second coil guide disposed to face the first coil guide, and a magnetic core disposed between the first coil guide and the second coil guide and including a coil wound thereon, and a method for operating the same.

### BACKGROUND

Allergic rhinitis is one of the most common diseases with increasing frequency of occurrence these days and is known as a disease that causes chronic pain in patients. Allergic rhinitis is treated with various drugs, but despite drug treatment, allergic rhinitis remains refractory in some patients, or relapses occurs frequently in patients treated with drugs. Related methods of treating the allergic rhinitis can be broadly classified into allergen avoidance therapy which reduces exposure to the cause as much as possible, drug therapy, immunotherapy, surgery, and the like, and while the most reliable therapy is to fundamentally block the invasion of antigens, there is a problem that it is difficult to fundamentally eliminate exposure to antigens because allergens that cause the allergic rhinitis are present widely in our life.

Based on this problem, an apparatus for treating rhinitis that is easy to carry and convenient to use and that treats inflammation by irradiating light in a specific wavelength band into the nasal cavity has been developed. However, when treating allergic rhinitis with such a related rhinitis therapy apparatus, since this involves an invasive method of directly inserting the apparatus into the nose for the treatment of allergic rhinitis, hygiene problems occur, and, while the light is used for the rhinitis therapy, there is a problem that the light can not penetrate deep enough into the nasal bone which is one of the regions where inflammation occurs in the nasal cavity, and as a result, the effect of the rhinitis therapy is insignificant, making it difficult to perform physical therapy.

### SUMMARY

In order to solve the problems described above, the present disclosure provides a rhinitis therapy apparatus and a method for operating the same.

According to the present disclosure, with a rhinitis therapy apparatus that is convenient to carry and easy to use, it is possible to perform a rhinitis therapy with simple operation.

In addition, according to the present disclosure, it is possible to effectively perform the rhinitis therapy by irradiating a magnetic field to the nose region of the user through the magnetic field focusing portion that is in the form of disc or protrusion formed on one side of the magnetic field generator of the rhinitis therapy apparatus.

According to the present disclosure, the rhinitis therapy apparatus can treat rhinitis by a non-invasive method rather than an invasive method of directly inserting the apparatus into the nose, and can be used semi-permanently without requiring cleaning or replacement of consumables.

According to the present disclosure, unlike the related LED/laser type rhinitis therapy apparatus, it is possible to treat rhinitis by transferring effective energy from an outside to an inside of a nasal cavity, without the need to insert the rhinitis therapy apparatus into the nose, by using a pulsed electromagnetic field (PEMF) with high biotransmittance.

According to the present disclosure, by using a low-frequency pulsed electromagnetic field of mT level, there is no side effect such as heat, glare, pain, and the like, and the rhinitis therapy apparatus does not block the view or block the nose, so that it can be used conveniently in daily life.

According to the present disclosure, the magnetic field focused from the point unit formed in the coil guide is irradiated toward the nose region of the user such that intensive treatment for rhinitis is enabled and the effect of the therapy is increased.

According to the present disclosure, the coil guide is made of the same material as the magnetic core, so that the irradiation range of the magnetic field is widened, thereby increasing the effect of the rhinitis therapy.

According to the present disclosure, by forming one of the plurality of coil guides with a wider diameter than the other, it is possible to reduce the material cost for the coil guides as compared to the case of forming the same diameter and achieve the same effect of the therapy.

The present disclosure may be implemented in various ways, including an apparatus, a method, or a computer-readable storage medium storing instructions.

The rhinitis therapy apparatus according to an embodiment may include a body configured to cover a nose region of a user, and a magnetic field generator provided on the body and irradiating a magnetic field toward the nose region, and the magnetic field generator may include a first coil guide, a second coil guide disposed to face the first coil guide, and a magnetic core disposed between the first coil guide and the second coil guide and including a coil wound thereon.

According to an embodiment, at least one of the first coil guide and the second coil guide may be made of the same material as the magnetic core.

According to an embodiment, one of the first coil guide and the second coil guide may be formed to be wider than the other.

According to an embodiment, the body may include the first side unit provided on one side of the body and the second side unit provided on the other side of the body, and the magnetic field generator may include the first magnetic field generator provided on the first side unit and the second magnetic field generator provided on the second side unit.

According to an embodiment, when the first magnetic field generator is controlled to generate a first pulsed magnetic field stimulation, the second magnetic field generator may generate a second pulsed magnetic field stimulation different from the first pulsed magnetic field stimulation, and according to a preset period, the first magnetic field generator and the second magnetic field generator may alternately generate the first pulsed magnetic field stimulation and the second pulsed magnetic field stimulation, respectively.

According to another embodiment of the present disclosure, a method for operating a rhinitis therapy apparatus is provided, which may include controlling, by a control unit, an operation of a magnetic field generator to irradiate a magnetic field toward a nose region of a user, and generating a magnetic field by the magnetic field generator and performing a rhinitis therapy for the nose region, in which the magnetic field generator may include a first coil guide, a second coil guide disposed to face the first coil guide, and a magnetic core disposed between the first coil guide and the second coil guide and including a coil wound thereon.

According to an embodiment, at least one of the first coil guide and the second coil guide may be made of the same material as the magnetic core.

According to an embodiment, one of the first coil guide and the second coil guide may be formed to be wider than the other.

According to an embodiment, the body may include the first side unit provided on one side of the body and the second side unit provided on the other side of the body, and the magnetic field generator may include the first magnetic field generator provided on the first side unit and the second magnetic field generator provided on the second side unit.

According to an embodiment, the operation of performing a rhinitis therapy for the nose region may include, when the first magnetic field generator generates the first pulsed magnetic field stimulation, generating, by the second magnetic field generator, the second pulsed magnetic field stimulation different from the first pulsed magnetic field stimulation, and by the first magnetic field generator and the second magnetic field generator, alternately generating the first pulsed magnetic field stimulation and the second pulsed magnetic field stimulation according to a preset period.

According to still another embodiment of the present disclosure, a computer-readable storage medium storing a program including one or more instructions for performing the method of operating the rhinitis therapy apparatus may be provided.

According to various embodiments of the present disclosure, the rhinitis therapy apparatus can treat rhinitis by a non-invasive method rather than an invasive method of directly inserting the apparatus into the nose, and can be used semi-permanently without requiring cleaning or replacement of consumables.

According to various embodiments of the present disclosure, unlike the related LED/laser type rhinitis therapy apparatus, it is possible to treat rhinitis by transferring effective energy from an outside to an inside of a nasal cavity, without the need to insert the rhinitis therapy apparatus into the nose, by using a pulsed electromagnetic field (PEMF) with high biotransmittance.

According to various embodiments of the present disclosure, by using a low-frequency pulsed electromagnetic field of mT level, there is no side effect such as heat, glare, pain, and the like, and the rhinitis therapy apparatus does not block the view or block the nose, so that it can be used conveniently in daily life.

According to various embodiments of the present disclosure, the magnetic field focused from the point unit formed in the coil guide is irradiated toward the nose region of the user such that intensive treatment for rhinitis is enabled and the effect of the therapy is increased.

According to various embodiments of the present disclosure, the coil guide is made of the same material as the magnetic core, so that the irradiation range of the magnetic field is widened, thereby increasing the effect of the rhinitis therapy.

According to various embodiments of the present disclosure, by forming one of the plurality of coil guides with a wider diameter than the other, it is possible to reduce the material cost for the coil guides as compared to the case of forming the same diameter and achieve the same effect of the therapy.

### BRIEF DESCRIPTION OF THE DRAWING

Embodiments of the present disclosure will be described with reference to the accompanying drawings described below, where similar reference numerals indicate similar elements, but embodiments are not limited thereto, in which:

FIG. 1 illustrates a user wearing a rhinitis therapy apparatus according to an embodiment;

FIG. 2 is an exemplary view illustrating the rhinitis therapy apparatus according to an embodiment;

FIG. 3 is a block diagram illustrating a configuration of the rhinitis therapy apparatus according to an embodiment;

FIG. 4 is an exemplary view illustrating a magnetic field generator included in the rhinitis therapy apparatus according to an embodiment;

FIG. 5 is a schematic diagram illustrating a configuration of the magnetic field generator according to an embodiment;

FIGS. 6A to 6D are exemplary views illustrating various examples of a point unit formed in a magnetic field generator according to an embodiment;

FIGS. 7A to 7C are exemplary views illustrating various examples of materials forming the magnetic field generator according to an embodiment;

FIGS. 8A to 8C are exemplary views illustrating examples of configurations of a side of the magnetic field generator having various widths according to an embodiment;

FIG. 9 is an exemplary view illustrating various examples of magnetic field stimulation mode used in the rhinitis therapy apparatus according to an embodiment; and

FIG. 10 shows a flowchart of a method for performing a rhinitis therapy according to an embodiment.

### DETAILED DESCRIPTION

Hereinafter, specific details for the practice of the present disclosure will be described in detail with reference to the accompanying drawings. However, in the following description, detailed descriptions of well-known functions or configurations will be omitted when it may make the subject matter of the present disclosure rather unclear.

In the accompanying drawings, the same or corresponding elements are assigned the same reference numerals. In addition, in the following description of the embodiments, duplicate descriptions of the same or corresponding components may be omitted. However, even if descriptions of elements are omitted, it is not intended that such elements are not included in any embodiment.

Advantages and features of the disclosed embodiments and methods of accomplishing the same will be apparent by referring to embodiments described below in connection with the accompanying drawings. However, the present disclosure is not limited to the embodiments disclosed below, and may be implemented in various different forms, and the embodiments are merely provided to make the present disclosure complete, and to fully disclose the scope of the invention to those skilled in the art to which the present disclosure pertains.

The terms used herein will be briefly described prior to describing the disclosed embodiments in detail. The terms used herein have been selected as general terms which are widely used at present in consideration of the functions of the present disclosure, and this may be altered according to the intent of an operator skilled in the art, conventional practice, or introduction of new technology. In addition, in specific cases, the term may be arbitrarily selected by the applicant, and the meaning of the term will be described in detail in a corresponding description of the embodiments. Therefore, the terms used in the present disclosure should be defined based on the meaning of the terms and the overall contents of the present disclosure rather than a simple name of each of the terms.

As used herein, the singular forms ‘a,’ ‘an,’ and ‘the’ are intended to include the plural forms as well, unless the context clearly indicates the singular forms. Further, the plural forms are intended to include the singular forms as well, unless the context clearly indicates the plural forms. Further, throughout the description, when a portion is stated as “comprising (including)” a component, it intends to mean that the portion may additionally comprise (or include or have) another component, rather than excluding the same, unless specified to the contrary.

Further, the term “module” or “unit” used herein refers to a software or hardware component, and “module” or “unit” performs certain roles. However, the meaning of the “module” or “unit” is not limited to software or hardware. The “module” or “unit” may be configured to be in an addressable storage medium or configured to reproduce one or more processors. Accordingly, as an example, the “module” or “unit” may include components such as software components, object-oriented software components, class components, and task components, and at least one of processes, functions, attributes, procedures, sub-routines, program code segments of program code, drivers, firmware, micro-codes, circuits, data, database, data structures, tables, arrays, or variables. Furthermore, functions provided in the components and the “modules” or “units” may be combined into a smaller number of components and “modules” or “units”, or further divided into additional components and “modules” or “units.”

According to an embodiment, the “module” or “unit” may be implemented as a processor and a memory. The “processor” should be interpreted broadly to encompass a general-purpose processor, a central processing unit (CPU), a microprocessor, a digital signal processor (DSP), a controller, a microcontroller, a state machine, and so forth. Under some circumstances, the “processor” may refer to an application-specific integrated circuit (ASIC), a programmable logic device (PLD), a field-programmable gate array (FPGA), and so on. The “processor” may refer to a combination of processing devices, e.g., a combination of a DSP and a microprocessor, a plurality of microprocessors, one or more microprocessors in conjunction with a DSP core, or any other combination of such configurations. In addition, the “memory” should be interpreted broadly to encompass any electronic component that is capable of storing electronic information.

In the present disclosure, a “diameter” may mean a diameter of a disk shape in general, and may be broadly interpreted as meaning the width and/or breadth of at least one side or part of components or parts of various shapes included in the rhinitis therapy apparatus.

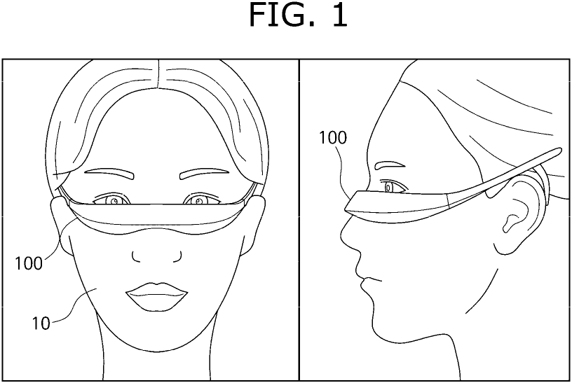

FIG. 1 illustrates a user 10 wearing a rhinitis therapy apparatus 100 according to an embodiment. The rhinitis therapy apparatus 100 is an apparatus capable of treating and/or improving allergic rhinitis, and may be mounted on the face of the user 10 for a rhinitis therapy. The rhinitis therapy apparatus 100 mounted on the face of the user 10 may perform a rhinitis therapy by irradiating a magnetic field to a nose region of a user.

In an embodiment, the rhinitis therapy apparatus 100 may be disposed in such a position where it 100 can straddle the nose bridge of the user 10 and cover the regions under both eyes of the user 10, as illustrated in FIG. 1. The rhinitis therapy apparatus 100 disposed as described above may generate a magnetic field toward the nose region to non-invasively treat rhinitis. With this configuration, the rhinitis therapy apparatus 100 can treat rhinitis by a non-invasive method rather than an invasive method of directly inserting the apparatus into the nose, and can be used semi-permanently without requiring cleaning or replacement of consumables.

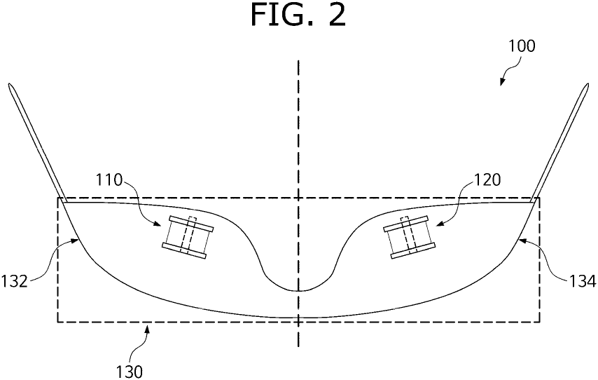

FIG. 2 is an exemplary view illustrating the internal configuration of the rhinitis therapy apparatus 100 according to an embodiment. Hereinafter, the components of the rhinitis therapy apparatus 100 will be described in more detail. As illustrated in FIG. 2, the rhinitis therapy apparatus 100 may include a body 130, a first side unit 132, a second side unit 134, a first magnetic field generator 110, and a second magnetic field generator 120. The body 130 may include two side units 132 and 134 formed on both sides with respect to the center line of the body 130 so as to be mounted on the face of the user 10 and cover the nose region of the user 10. The two side units 132 and 134 may include the first side unit 132 provided on one side of the center line and the second side unit 134 provided on the other side of the center line. The magnetic field generator may include the first magnetic field generator 110 and the second magnetic field generator 120, which may be included in the first side unit 132 and the second side unit 134, respectively. The first magnetic field generator 110 and the second magnetic field generator 120 may irradiate a magnetic field under the control of a control unit (not illustrated).

In an embodiment, the body 130 of the rhinitis therapy apparatus 100 may have a curved shape to be naturally seated along the contour of the nose region of the user 10, and be disposed at such a position where it 130 straddles the bridge of the nose and covers not only the nose region of the user 10, but also the regions under both eyes. Specifically, when the body 130 is brought into contact with the user’s skin, the first side unit 132 and the second side unit 134 formed on the body may come into contact with positions corresponding to the regions under both eyes of the user, respectively. The first magnetic field generator 110 and the second magnetic field generator 120 respectively included in the first side unit 132 and the second side unit 134 may be configured to irradiate a magnetic field from the regions under the eyes of the user toward the nose region where the nasal cavity is located. For example, by the control unit, the first magnetic field generator 110 may irradiate a magnetic field from the region under the left eye to the left nose region of the user, and the second magnetic field generator 120 may irradiate a magnetic field from the region under the right eye to the right nose region of the user such that a rhinitis therapy is performed.

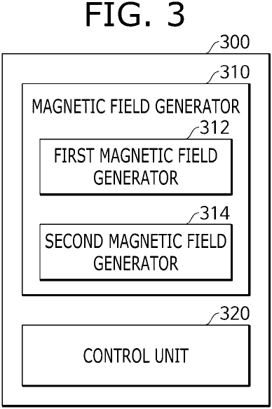

FIG. 3 is a schematic diagram illustrating a configuration of the rhinitis therapy apparatus according to an embodiment. The rhinitis therapy apparatus 100 may include a magnetic field generator 310 and a control unit 320, and the magnetic field generator 310 may include a first magnetic field generator 312 and a second magnetic field generator 314. The control unit 320 may control the first magnetic field generator 312 and the second magnetic field generator 314 to irradiate a magnetic field for rhinitis therapy. When the control unit 320 controls the first magnetic field generator 312 to generate a first pulsed magnetic field stimulation, the control unit 320 may control the second magnetic field generator 314 to generate a second pulsed magnetic field stimulation different from the first pulsed magnetic field stimulation. In addition, the control unit 300 may control the first magnetic field generator 312 and the second magnetic field generator 314 to alternately generate the first pulsed magnetic field stimulation and the second pulsed magnetic field stimulation according to a preset period. In an embodiment, the control unit 300 may control the magnetic field generators 312 and 314 to generate one of an N pulse stimulation mode, an S pulse stimulation mode, an alternating stimulation mode of N pulses and S pulses, an N pulse continuous stimulation mode, and an S pulse continuous stimulation mode. For example, after controlling the first magnetic field generator 312 to irradiate the magnetic field corresponding to the N pulse stimulation mode toward the region under the left eye of the user 10, the control unit 300 of the rhinitis therapy apparatus 100 may control the second magnetic field generator 314 to irradiate a magnetic field corresponding to the S pulse stimulation mode toward the region under the right eye of the user 10. The magnetic fields irradiated through the first magnetic field generator 312 and the second magnetic field generator 314 may provide a stimulation related to promoting blood ionization to the nose region and/or regions under the eyes of the user 10 to treat rhinitis of the user 10. With this configuration, unlike the related LED/laser type rhinitis therapy apparatus, it is possible to treat rhinitis by transferring effective energy from an outside to an inside of a nasal cavity, without the need to insert the rhinitis therapy apparatus into the nose, by using a pulsed electromagnetic field (PEMF) with high biotransmittance.

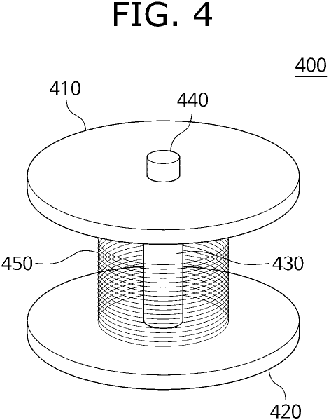

FIG. 4 is an exemplary view illustrating a magnetic field generator 400 included in the rhinitis therapy apparatus according to an embodiment. The magnetic field generator 400 may be included in the first magnetic field generator 110 and 312 or the second magnetic field generator 120 and 314 illustrated in FIGS. 2 and 3.

As illustrated, the magnetic field generator 400 may include a first coil guide 410, a second coil guide 420, a magnetic core 430, a point unit 440, and a coil 450. The first coil guide 410 and the second coil guide 420 may be formed at opposing positions to face each other. The magnetic core 430 may be positioned between the first coil guide 410 and the second coil guide 420 so as to be orthogonal to each of the first coil guide 410 and the second coil guide 420, and the coil 450 may be wound around the side of the magnetic core 430.

In an embodiment, the magnetic field generator 400 may be an electromagnet including the magnetic core 430 and the coil 450. For example, the coil 450 may be a solenoid coil. The protruding point unit 440 may be formed on one side of the magnetic field generator 400 to focus the magnetic field generated through the corresponding side to a predetermined direction. For example, the protruding point unit 440 may be formed on one of the first coil guide 410 and the second coil guide 420.

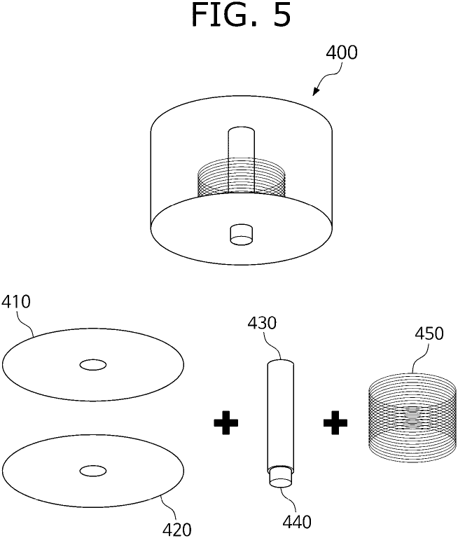

FIG. 5 is a schematic diagram illustrating a configuration of the magnetic field generator according to an embodiment. Hereinafter, the components of the magnetic field generator 400 will be described in more detail. The magnetic field generator 400 may include two coil guides 410 and 420, the magnetic core 430 including the point unit 440 formed at one end, and the coil 450. A central hole is formed in each of the first coil guide 410 and the second coil guide 420 to allow coupling with the magnetic core 430 having the point unit 440 formed thereon. The point unit 440 may be configured such that, upon coupling of the magnetic core 430 with the first coil guide 410 and the second coil guide 420, the point unit 440 may protrude through one of the first coil guide 410 and the second coil guide 420. When the coupling of the coil guides 410 and 420 and the magnetic core 430 is completed, the coil 450 may be wound around the side of the magnetic core 430 positioned between the coil guides 410 and 420. The magnetic field generator 400 may be provided in each of the first side unit 132 and the second side unit 134 of the rhinitis therapy apparatus 100 with the point units 440 being positioned under the eyes of the user 10 so as to irradiate a magnetic field from the regions under the eyes of the user 10 toward the nose region where the nasal cavity is located. For example, the magnetic field generated from a magnetic field generator 200 may be a pulsed electromagnetic field (PEMF). The magnetic field generated from the magnetic field generator 400 may be a static electromagnetic field or a weak magnetic field having a size of about 1 Gauss to about 320 Gauss. In another example, the magnetic flux density of the magnetic field may be in the range of 0 mT to 100 mT (1000 Gauss). With this configuration, by using a low-frequency pulsed electromagnetic field of mT level, there is no side effect such as heat, glare, pain, and the like, and the rhinitis therapy apparatus does not block the view or block the nose, so that it can be used conveniently in daily life.

In the embodiment described above, it is described that the coil guides 410 and 420 and the magnetic core 430 are separately manufactured and then assembled, but the embodiment is not limited thereto, and the coil guides 410 and 420 and the magnetic core 430 may be integrally formed.

In an embodiment, one of the first coil guide 410 and the second coil guide 420 disposed to face each other may be formed of the same material as the magnetic core 430 and the point unit 440. For example, the first coil guide 410, the second coil guide 420, the magnetic core 430, and the point unit 440 may be formed of S45C (carbon steel) material, which is a ferromagnetic material having a strong magnetization force, to expand the range of the magnetic field formed from the magnetic field generator 400, thereby increasing the effect of the rhinitis therapy.

For the rhinitis therapy, the stimulation source generated from the magnetic field generator 400 may be an electromagnetic field, and the magnetic field generator 400 may generate a static electromagnetic field or a time-varying electromagnetic field (0 kHz to 1 kHz) of a low frequency region. For example, the static electromagnetic field may refer to a magnetic field generated by applying a constant current (DC) to the magnetic field generator 400, and the time-varying magnetic field may refer to a magnetic field generated by applying a pulse or sine wave current.

In addition, the magnetic field generator 400 may generate various types of magnetic field stimulation. For example, the type of magnetic field generated by the magnetic field generator 400 may include at least one of a sine wave, a square wave, and a pulse wave. The operation of the magnetic field generator 400 may be controlled by the control of the control unit 320. For example, at least one of the strength (magnetic flux density), frequency, time, and pattern (pulse form) of the magnetic field generated from the magnetic field generator 400 may be controlled by the control of the control unit 320.

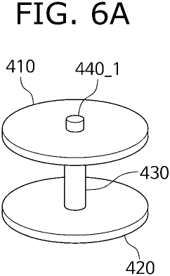

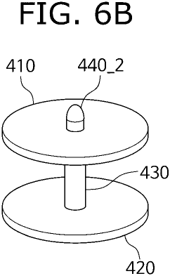

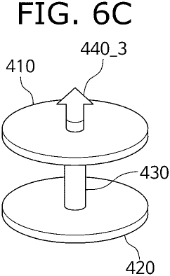

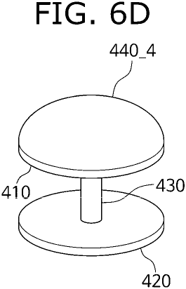

FIGS. 6A to 6D are exemplary views illustrating various examples of point units 440_1, 440_2, 440_3, and 440_4 formed in the magnetic field generator according to an embodiment. The point unit 440 in the form of a projection may be formed on one side of the magnetic field generator 400 to focus the magnetic field generated from the magnetic field generator 400.

For example, the point unit 440 in cylindrical shape (FIG. 6A), cone shape (FIG. 6B), arrow shape (FIG. 6C), or the like may be integrally formed with one of the first coil guide 410 and the second coil guide 420. In another example, the point unit 440 in cylindrical shape (FIG. 6A), cone shape (FIG. 6B), arrow shape (FIG. 6C), or the like may be integrally formed with the magnetic core 430 and then coupled to the first coil guide 410 and the second coil guide 420 which have the central hole formed therein. In still another example, one of the first coil guide 410 and the second coil guide 420 may be formed in a convex shape (e.g., in the shape of a hemisphere illustrated in FIG. 6D) to be able to focus the magnetic field and serve as the point unit 440.

In an embodiment, the magnetic field generators 400 may be disposed on the first side unit 132 and the second side unit 134 such that the point units 440 may face the user’s face when the user 10 wears the rhinitis therapy apparatus 100. In this case, the point unit 440 may focus the magnetic field on the local area where the nasal cavity is located to increase the effect of non-invasive rhinitis therapy. That is, the magnetic field focused from the point unit formed in the coil guide is irradiated toward the nose region of the user such that intensive treatment for rhinitis is enabled and the effect of the therapy is increased.

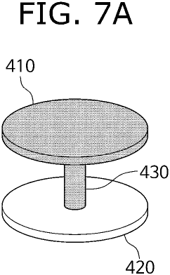

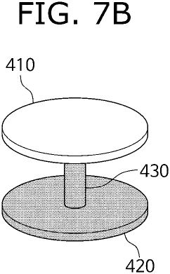

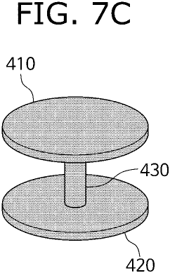

FIGS. 7A to 7C are exemplary views illustrating various examples of materials forming the magnetic field generator according to an embodiment. At least one of the first coil guide 410 and the second coil guide 420 may be made of the same material as the magnetic core 430.

In an embodiment, as illustrated in FIG. 7A, the first coil guide 410 may be made of the same material as the magnetic core 430. In another embodiment, as illustrated in FIG. 7B, the second coil guide 420 may be made of the same material as the magnetic core 430. In still another embodiment, as illustrated in FIG. 7C, the first coil guide 410 and the second coil guide 420 may be made of the same material as the magnetic core 430. For example, the magnetic core 430 may be a ferromagnetic material having a strong magnetization force, and may be made of a ferrite and/or S45C (carbon steel) material. When one or more of the first coil guide 410 and the second coil guide 420 are made of the same ferromagnetic material as the magnetic core 430, the irradiation range of the magnetic field is broader than when only the magnetic core 430 is made of the ferromagnetic material, thereby increasing the effect of the rhinitis therapy.

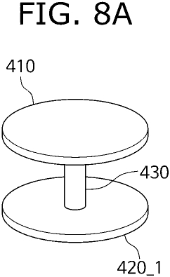

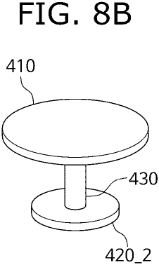

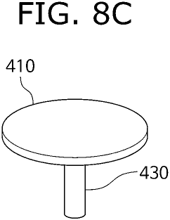

FIGS. 8A to 8C are exemplary views illustrating examples of configurations of the side of the magnetic field generator having various widths according to an embodiment. One of the first coil guide 410 and the second coil guide 420 may be formed to be wider than the other coil guide. In an embodiment, the first coil guide 410 may have the same diameter as the second coil guide 420 or may have a diameter greater than that of the second coil guide 420.

For example, as illustrated in FIG. 8A, the first coil guide 410 may have the same diameter as the second coil guide 420_1. In another example, as illustrated in FIG. 8B, the first coil guide 410 may have a diameter greater than that of the second coil guide 420_2. In another example, as illustrated in FIG. 8C, the second coil guide may not be formed in the magnetic field generator, and the magnetic core 430 may serve as the second coil guide instead. As described above, by way of configuring the first coil guide 410 and the second coil guide 420 with different diameters, it is possible to reduce the material cost for the coil guides as compared to when configuring them with the same diameter, and also perform effective treatment.

For example, as illustrated in FIG. 8B, when the magnetic field generator is disposed in the rhinitis therapy apparatus in such a way that the first coil guide 410 formed wider than the second coil guide 420_2 faces the nose region, the effect of rhinitis therapy can be increased. Meanwhile, by making a smaller second coil guide 420_2 which does not face the nose region, the material cost required for manufacturing the magnetic field generator can be reduced.

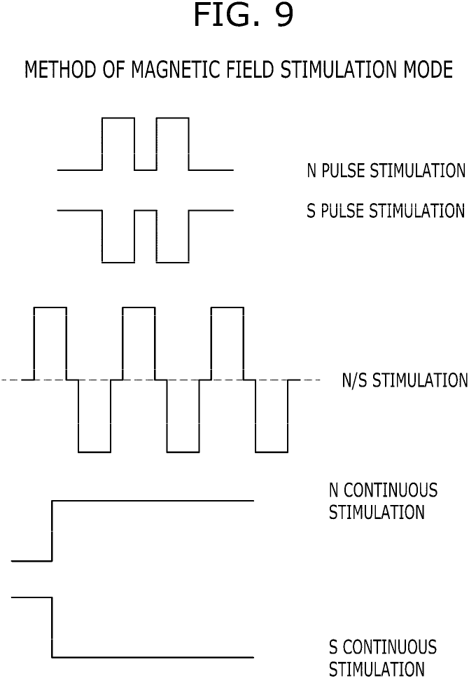

FIG. 9 is an exemplary view illustrating various examples of magnetic field stimulation mode used in the rhinitis therapy apparatus according to an embodiment. The control unit 320 may control the magnetic field stimulation mode generated from the magnetic field generator 400. The magnetic field stimulation mode may include at least one of an N pulse stimulation mode, an S pulse stimulation mode, an alternating stimulation (N/S stimulation) mode of N pulses and S pulses, an N pulse continuous stimulation (N continuous stimulation) mode, and an S pulse continuous stimulation (S continuous stimulation) mode. For example, the magnetic field generator 400 may generate N pulsed magnetic field stimulation by irradiating the magnetic field corresponding to the N pulse stimulation mode, and generate S pulsed magnetic field stimulation by irradiating the magnetic field corresponding to the S pulse stimulation mode.

In another example, the magnetic field generator 400 may generate N/S alternating magnetic field stimulation by irradiating the magnetic field corresponding to the alternating stimulation mode of N pulses and S pulses. In another example, the magnetic field generator 200 may generate N pulse continuous magnetic field stimulation by irradiating the magnetic field corresponding to the N pulse continuous stimulation mode, and generate S pulse continuous magnetic field stimulation by irradiating the magnetic field corresponding to the S pulse continuous stimulation mode.

The magnetic field generator 400 may irradiate a magnetic field to the nose region and/or regions under the eyes of the user 10, so that the magnetic field may be applied to blood corresponding to the nose region and/or regions under the eyes. As a result, oxygen (O₂) binds to iron (Fe) in the heme group in hemoglobin of red blood cells in blood corresponding to the nose region and/or regions under the eyes, thereby promoting blood ionization. In an embodiment, the magnetic field generator 400 may generate pulsed electromagnetic field (PEMF) under the control of the control unit 320. Through the pulsed electromagnetic field (PEMF), an alternating magnetic force (Lorentz force) may be applied to iron ions in the hemoglobin of red blood cells in the blood corresponding to the nose region and/or regions under the eyes of the user 10, and accordingly, red blood cells in a state of rouleaux are separated freely, so that the phenomenon of rouleaux is improved, and blood ionization and the like can be promoted.

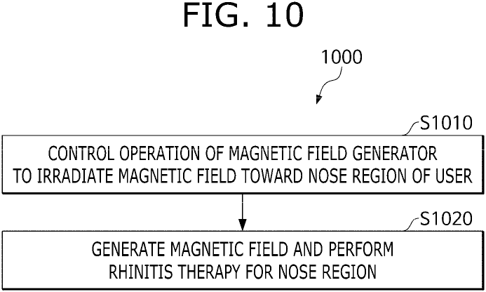

FIG. 10 shows a flowchart of a method for performing a rhinitis therapy according to an embodiment. A method 1000 for performing a rhinitis therapy may be initiated by controlling the operation of the magnetic field generator to irradiate the magnetic field toward the nose region of the user, at S1010.

In an embodiment, the control unit 320 may control the magnetic field generator 400 according to an input of the user 10. For example, the control unit may control the strength, frequency, time, pattern, and the like of the magnetic field for the type of magnetic field generated from the magnetic field generator 400. Then, it is possible to generate a magnetic field to perform a rhinitis therapy for the nose region, at S1020. In an embodiment, the magnetic field generated by the magnetic field generator 400 is irradiated toward the nose region and/or regions under the eyes of the user, so that rhinitis therapy for the nose region of the user can be performed.

In an embodiment, at least one of the first coil guide and the second coil guide may be made of the same material as the magnetic core. In addition, one of the first coil guide and the second coil guide may be formed to be wider than the other.

With this configuration, the coil guide is made of the same material as the magnetic core, so that the irradiation range of the magnetic field is widened, thereby increasing the effect of the rhinitis therapy.

In addition, by forming one of the plurality of coil guides with a wider diameter than the other, it is possible to reduce the material cost for the coil guides as compared to the case of forming the same diameter and achieve the same effect of the therapy.

In an embodiment, the body may include the first side unit provided on one side of the body and the second side unit provided on the other side of the body, and the magnetic field generator may include the first magnetic field generator provided on the first side unit and the second magnetic field generator provided on the second side unit.

In an embodiment, the operation of performing a rhinitis therapy for the nose region may include, when the first magnetic field generator generates the first pulsed magnetic field stimulation, generating, by the second magnetic field generator, the second pulsed magnetic field stimulation different from the first pulsed magnetic field stimulation, and, by the first magnetic field generator and the second magnetic field generator, alternately generating the first pulsed magnetic field stimulation and the second pulsed magnetic field stimulation according to a preset period.

With this configuration, unlike the related LED/laser type rhinitis therapy apparatus, it is possible to treat rhinitis by transferring effective energy from an outside to an inside of a nasal cavity, without the need to insert the rhinitis therapy apparatus into the nose, by using a pulsed electromagnetic field (PEMF) with high biotransmittance.

In addition, by using a low-frequency pulsed electromagnetic field of mT level, there is no side effect such as heat, glare, pain, and the like, and the rhinitis therapy apparatus does not block the view or block the nose, so that it can be used conveniently in daily life.

Although the present disclosure has been described in connection with some embodiments herein, it should be understood that various modifications and changes can be made without departing from the scope of the present disclosure, which can be understood by those skilled in the art to which the present disclosure pertains. Further, such modifications and changes are intended to fall within the scope of the claims appended herein.

In addition, the methods, operations, or techniques of the present disclosure may be implemented by various means. For example, these techniques may be implemented in hardware, firmware, software, or a combination thereof. Those skilled in the art will further appreciate that various illustrative logical blocks, modules, circuits, and algorithm steps described in connection with the disclosure herein may be implemented in electronic hardware, computer software, or combinations of both.

To clearly illustrate this interchangeability of hardware and software, various illustrative components, blocks, modules, circuits, and steps have been described above generally in terms of their functionality. Whether such a function is implemented as hardware or software varies depending on design requirements imposed on the particular application and the overall system. Those skilled in the art may implement the described functions in varying ways for each particular application, but such implementation should not be interpreted as causing a departure from the scope of the present disclosure.

In a hardware implementation, processing units used to perform the techniques may be implemented in one or more ASICs, DSPs, digital signal processing devices (DSPDs), programmable logic devices (PLDs), field programmable gate arrays (FPGAs), processors, controllers, microcontrollers, microprocessors, electronic devices, other electronic units designed to perform the functions described in the disclosure, computer, or a combination thereof.

Accordingly, various example logic blocks, modules, and circuits described in connection with the disclosure may be implemented or performed with general purpose processors, DSPs, ASICs, FPGAs or other programmable logic devices, discrete gate or transistor logic, discrete hardware components, or any combination of those designed to perform the functions described herein. The general purpose processor may be a microprocessor, but in the alternative, the processor may be any related processor, controller, microcontroller, or state machine. The processor may also be implemented as a combination of computing devices, for example, a DSP and microprocessor, a plurality of microprocessors, one or more microprocessors associated with a DSP core, or any other combination of the configurations.

In the implementation using firmware and/or software, the techniques may be implemented with instructions stored on a computer-readable medium, such as random access memory (RAM), read-only memory (ROM), non-volatile random access memory (NVRAM), programmable read-only memory (PROM), erasable programmable read-only memory (EPROM), electrically erasable PROM (EEPROM), flash memory, compact disc (CD), magnetic or optical data storage devices, and the like. The instructions may be executable by one or more processors, and may cause the processor(s) to perform certain aspects of the functions described in the present disclosure.

When implemented in software, the techniques may be stored on a computer-readable medium as one or more instructions or codes, or may be transmitted through a computer-readable medium. The computer-readable media include both the computer storage media and the communication media including any medium that facilitates the transfer of a computer program from one place to another.

The storage media may also be any available media that may be accessed by a computer. By way of non-limiting example, such a computer-readable medium may include RAM, ROM, EEPROM, CD-ROM or other optical disk storage, magnetic disk storage or other magnetic storage devices, or any other media that can be used to transfer or store desired program code in the form of instructions or data structures and can be accessed by a computer. Also, any connection is properly referred to as a computer-readable medium.

For example, when the software is transmitted from a website, server, or other remote sources using coaxial cable, fiber optic cable, twisted pair, digital subscriber line (DSL), or wireless technologies such as infrared, wireless, and microwave, the coaxial cable, the fiber optic cable, the twisted pair, the digital subscriber line, or the wireless technologies such as infrared, wireless, and microwave are included within the definition of the medium.

The disks and the discs used herein include CDs, laser disks, optical disks, digital versatile discs (DVDs), floppy disks, and Blu-ray disks, where disks usually magnetically reproduce data, while discs optically reproduce data using a laser. The combinations described above should also be included within the scope of the computer-readable media.

The software module may reside in RAM memory, flash memory, ROM memory, EPROM memory, EEPROM memory, registers, hard disk, removable disk, CD-ROM, or any other form of storage medium known. An exemplary storage medium may be connected to the processor, such that the processor may read or write information from or to the storage medium. Alternatively, the storage medium may be integrated into the processor. The processor and the storage medium may exist in the ASIC. The ASIC may exist in the user terminal. Alternatively, the processor and storage medium may exist as separate components in the user terminal.

Although the embodiments described above have been described as utilizing aspects of the currently disclosed subject matter in one or more standalone computer systems, embodiments are not limited thereto, and may be implemented in conjunction with any computing environment, such as a network or distributed computing environment. Furthermore, the aspects of the subject matter in the present disclosure may be implemented in multiple processing chips or devices, and storage may be similarly influenced across a plurality of devices. Such devices may include PCs, network servers, and portable devices.

Although the present disclosure has been described in connection with some embodiments herein, various modifications and changes can be made without departing from the scope of the present disclosure, which can be understood by those skilled in the art to which the present disclosure pertains. Further, such modifications and changes are intended to fall within the scope of the claims appended herein.

1. A rhinitis therapy apparatus, comprising:
a body configured to cover a nose region of a user; and
a magnetic field generator provided on the body and irradiating a magnetic field toward the nose region,
wherein the magnetic field generator includes:
a first coil guide;
a second coil guide disposed to face the first coil guide; and
a magnetic core disposed between the first coil guide and the second coil guide and including a coil wound thereon.

2. The rhinitis therapy apparatus according to claim 1, wherein at least one of the first coil guide and the second coil guide is made of a same material as the magnetic core.

3. The rhinitis therapy apparatus according to claim 1, wherein one of the first coil guide and the second coil guide is formed to be wider than the other.

4. The rhinitis therapy apparatus according to claim 1, wherein the body includes:
a first side unit provided on one side of the body; and
a second side unit provided on the other side of the body, and
the magnetic field generator includes:
a first magnetic field generator provided on the first side unit; and
a second magnetic field generator provided on the second side unit.

5. The rhinitis therapy apparatus according to claim 1, wherein, when the first magnetic field generator is controlled to generate a first pulsed magnetic field stimulation, the second magnetic field generator generates a second pulsed magnetic field stimulation different from the first pulsed magnetic field stimulation, and according to a preset period, the first magnetic field generator and the second magnetic field generator alternately generate the first pulsed magnetic field stimulation and the second pulsed magnetic field stimulation, respectively.

6. A method for operating a rhinitis therapy apparatus, comprising:
controlling, by a control unit, an operation of a magnetic field generator to irradiate a magnetic field toward a nose region of a user; and
generating a magnetic field by the magnetic field generator and performing a rhinitis therapy for the nose region,
wherein the magnetic field generator includes:
a first coil guide;
a second coil guide disposed to face the first coil guide; and
a magnetic core disposed between the first coil guide and the second coil guide and including a coil wound thereon.

7. The method according to claim 6, wherein at least one of the first coil guide and the second coil guide is made of a same material as the magnetic core.

8. The method according to claim 6, wherein one of the first coil guide and the second coil guide is formed to be wider than the other.

9. The method according to claim 6, wherein the body includes:
a first side unit provided on one side of the body; and
a second side unit provided on the other side of the body, and
wherein the magnetic field generator includes:
a first magnetic field generator provided on the first side unit; and
a second magnetic field generator provided on the second side unit.

10. The method according to claim 6, wherein the performing a rhinitis therapy for the nose region includes:
when the first magnetic field generator generates a first pulsed magnetic field stimulation, generating, by the second magnetic field generator, a second pulsed magnetic field stimulation different from the first pulsed magnetic field stimulation; and
by the first magnetic field generator and the second magnetic field generator, alternately generating the first pulsed magnetic field stimulation and the second pulsed magnetic field stimulation according to a preset period.

11. A non-transitory computer readable medium storing a program including one or more instructions for performing the method according to claim 6.
